(19) United States
(12) Patent Application Publication
(10) Pub. No.: US 2016/0144166 A1
(43) Pub. Date: May 26, 2016

(54) MEDICAL LEAD WITH THIN FILM

(71) Applicant: MEDTRONIC BAKKEN RESEARCH CENTER B.V., MAASTRICHT (NL)

(72) Inventors: Michel Marcel José Decré, Eindhoven (NL); Johannes Wilhelmus Weekamp, Beek en Donk (NL); Egbertus Reinier Jacobs, Overloon (NL); Johannes van Roosmalen, Sint-Oedenrode (NL)

(21) Appl. No.: 14/951,237

(22) Filed: Nov. 24, 2015

Related U.S. Application Data
(60) Provisional application No. 62/084,378, filed on Nov. 25, 2014.

(51) Int. Cl. A61N 1/05; A61B 5/00; A61N 1/375; A61B 5/04

(52) U.S. Cl. CPC A61N 1/0534; A61B 5/04; A61B 5/686; A61N 1/3754; A61B 2562/125

(57) ABSTRACT

A medical lead includes an elongated carrier, a thin film attached to the elongated carrier, the thin film including a plurality of electrodes, a plurality of electrical contacts, and a plurality of conducting tracks, each of the plurality of conducting tracks providing an electrical connection between at least one of the plurality of electrodes and one of the plurality of electrical contacts; and a frame element including a fixation zone for the plurality of electrical contacts of the thin film.

This application claims the benefit of U.S. Provisional Application Serial No. 62/084,378, by Weekamp et al., and filed Nov. 25, 2014, which is incorporated herein by reference in its entirety.

### TECHNICAL FIELD

This disclosure relates, in some examples, to medical leads and medical device systems.

### BACKGROUND

Implantable neurostimulation devices may treat acute or chronic neurological conditions. Deep brain stimulation (DBS), which may include, e.g., the mild electrical stimulation of cortical and/or sub-cortical structures, belongs to this category of implantable devices, and has been shown to be therapeutically effective for such conditions as Parkinson's disease, Dystonia, Epilepsy, Alzheimer's Disease, and Tremor. As another example, DBS may be used to treat psychiatric disorders (obsessive-compulsive disorder, depression). DBS systems generally include one or more leads connected to an implantable pulse generator.

### SUMMARY

This disclosure is directed to medical leads including thin films incorporating both the electrodes of the medical leads and the conducting tracks for the medical leads.

In one example, this disclosure is directed to a medical device system for at least one of delivery of electrical stimulation pulses or sensing of physiological signals, the system comprising an elongated carrier; a thin film attached to the elongated carrier, the thin film including a plurality of electrodes, a plurality of electrical contacts, and a plurality of conducting tracks, each of the plurality of conducting tracks providing an electrical connection between at least one of the plurality of electrodes and one of the plurality of electrical contacts; and a frame element including a fixation zone for the plurality of electrical contacts of the thin film.

In another example, this disclosure is directed to a method of manufacturing a medical lead, the method comprising assembling a thin film to an elongated carrier, the thin film including a plurality of electrodes, a plurality of electrical contacts, and a plurality of conducting tracks, each of the plurality of conducting tracks providing an electrical connection between at least one of the plurality of electrodes and one of the plurality of electrical contacts, and fixing the plurality of electrical contacts of the thin film on a fixation zone of a frame element.

In another example, an implantable medical device including a first substantially sealed housing; an active lead can including a second substantially sealed housing operatively coupled to the implantable medical device; and a medical lead extending from the active lead can and operatively coupled to the active lead can. The medical lead includes an elongated carrier; a thin film attached to the elongated carrier, the thin film including a plurality of electrodes, a plurality of electrical contacts, and a plurality of conducting tracks, each of the plurality of conducting tracks providing an electrical connection between at least one of the plurality of electrodes and one of the plurality of electrical contacts; and a frame element including a fixation zone for the plurality of electrical contacts of the thin film.

The details of one or more examples of this disclosure may be set forth in the accompanying drawings and the description below. Other features, objects, and advantages of this disclosure may be apparent from the description and drawings, and from the claims.

### BRIEF DESCRIPTION OF DRAWINGS

FIG. 1 is a conceptual diagram illustrating an example deep brain stimulation (DBS) system configured to sense a bioelectrical brain signal and deliver electrical stimulation therapy to a tissue site within a brain of a patient.

FIG. 2 is a functional block diagram illustrating components of an example medical device system including an implantable pulse generator and a separate active lead can (ALC) with a switch matrix to direct signals from the implantable pulse generator to different electrodes.

FIG. 3 illustrates the electrical paths between an implantable pulse generator and a separate ALC with a switch matrix to direct signals from the implantable pulse generator to different electrodes.

FIGS. 4A-4C illustrate examples of medical leads for stimulation and/or sensing that may be used in the systems of FIGS. 1-3.

FIGS. 5-11 illustrate a proximal portion of a medical lead during manufacturing of the medical lead including forming a connection between a thin film element and an ALC of the medical lead.

FIG. 12 is a functional block diagram illustrating components of an example medical device programmer.

FIG. 13 is a flowchart illustrating example techniques for manufacturing a medical lead.

### DETAILED DESCRIPTION

This disclosure includes techniques for electrically and mechanically connecting a probe including electrodes and other components of a medical system configured to deliver therapy and/or provide sensing via the electrodes. For neural stimulation electrode arrays positioned inside the brain, a probe may include a flexible tube or other elongated carrier supporting a thin film containing electrodes and conducting tracks wrapped around the elongated carrier. Examples of this disclosure may be applied to, for example, leads for deep brain stimulation, cochlear implants, hearing aids, pacemakers, implantable cardiac defibrillators, and other implantable systems including stimulation and/or sensing leads connected to a control box.

In a further example, a medical lead may connect to an active lead can (ALC) mounted to a frame element. The lead may be coupled to electronics to transmit electrical stimulation current via selected electrodes. At least a part of the electronics of the lead may be arranged in the ALC, which may be a box-like structure. The active lead may be coupled to a proximal end of the lead, which, in turn, may be coupled via a lead extension to an implantable pulse generator (IPG). A mounting of the ALC to the frame element may provide option for improved connection of the electronics to the thin film, which may be supported and fixed on the frame element.

The ALC may contain at least a part of the electronics of the medical lead, with at least some of the electronics being connected to the thin film in the area of the fixation zone. In particular, the ALC may be hermetically or substantially hermetically sealed and may include connections to address the plurality of electrodes on the distal end of the thin film, which is arranged at the distal end and may be next to a distal tip of the lead. The plurality of electrodes may comprise any number of electrodes, and in one example contains approximately 40 electrodes. The electrodes may be arranged such that the electrodes are evenly distributed all over the distal end of the lead.

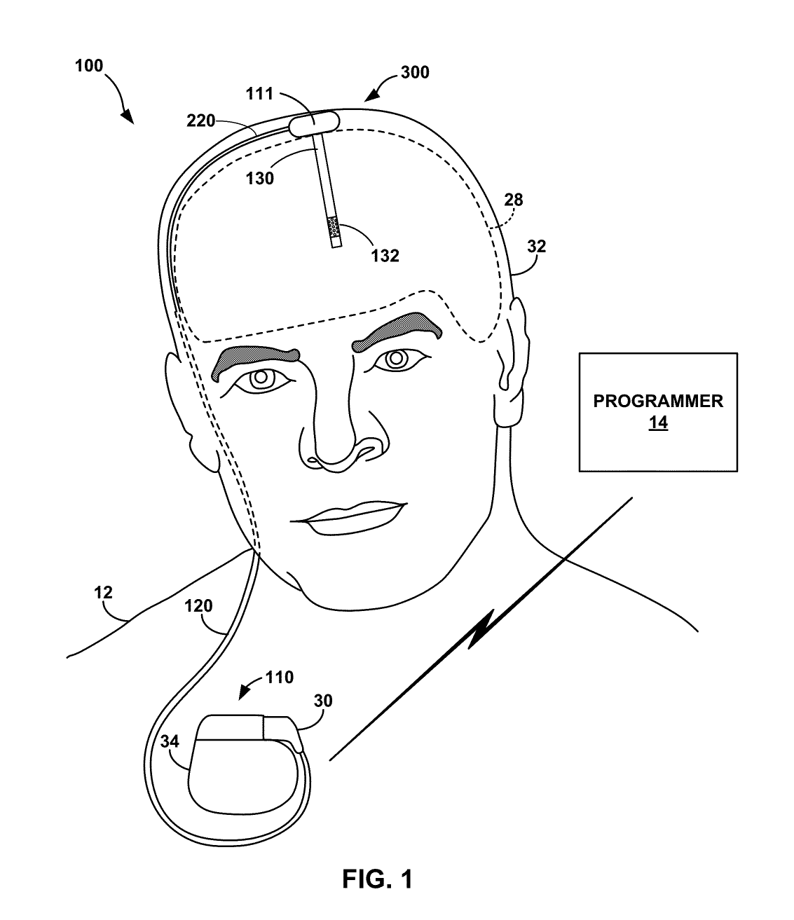

FIG. 1 is a conceptual diagram illustrating an example therapy system 100 that is configured to deliver therapy to patient 12 to manage a disorder of patient 12. Patient 12 ordinarily will be a human patient. In some cases, however, therapy system 100 may be applied to other mammalian or non-mammalian non-human patients. In the example shown in FIG. 1, therapy system 100 includes medical device programmer 14, IPG 110 (also referred to as an implantable medical device (IMD)), ALC 111, which connects to IPG 110 via lead extension 120 and lead 220. For example, the proximal end of lead extension 120 may be coupled to IPG 110 and at its distal end to the proximal end of lead 220 via an electrical connector(s) (not shown). Alternatively, a single lead may extend from IPG 110 to ALC 111. The distal end of lead 220 may be coupled to ALC 111 which is coupled to probe 130 with electrodes 132. IPG 110 may include at least one, such as two, stimulation generators configured to generate and deliver electrical stimulation therapy to one or more regions of brain 28 of patient 12 via one or more electrodes 132 of probe 130, respectively, alone or in combination with an electrode provided by outer housing 34 of IPG 110 and/or an electrode provided by the housing of ALC 111. In another example, any number of additional stimulation generators may be provided. Outer housing 34 of IPG 110 the housing of ALC 111 may each represent substantially sealed, such as hermetically sealed, housings containing electronics.

In the example shown in FIG. 1, therapy system 100 may be referred to as a DBS system because IPG 110 is configured to deliver electrical stimulation therapy directly to tissue within brain 28, for example, a tissue site under the dura mater of brain 28 or one or more branches or nodes, or a confluence of fiber tracks. In other examples, probe 130 may be positioned to deliver therapy to a surface of brain 28 (e.g., the cortical surface of brain 28). For example, in some examples, IPG 110 may provide cortical stimulation therapy to patient 12, for example, by delivering electrical stimulation to one or more tissue sites in the cortex of brain 28. Frequency bands of therapeutic interest in cortical stimulation therapy may include the theta band, and the gamma band.

DBS may be used to treat or manage various patient conditions, such as, but not limited to, seizure disorders (e.g., epilepsy), pain, migraine headaches, psychiatric disorders (e.g., major depressive disorder (MDD), bipolar disorder, anxiety disorders, post-traumatic stress disorder, dysthymic disorder, and obsessive-compulsive disorder (OCD), behavior disorders, mood disorders, memory disorders, mentation disorders, movement disorders (e.g., essential tremor or Parkinson's disease), Huntington's disease, Alzheimer's disease, or other neurological or psychiatric disorders and impairment of patient 12.

DBS leads may implement monopolar, bipolar, or even tripolar stimulation. Neurostimulator devices with steering brain stimulation capabilities may have a large number M of electrode contacts, such as M>10, M>20 and/or M=40, that may be connected to electrical circuits such as current sources and/or (system) ground. Even more electrodes may be provided in some examples. Stimulation may be considered monopolar when the distance between the anode and cathode is several times larger than the distance of the cathode to the stimulation target. During monopolar stimulation in homogeneous tissue, the electric field may be distributed roughly spherically similar to the field from a point source. When the anode is located close to the cathode, creating a bipolar electrode combination, the distribution of the field becomes more directed in the anode-cathode direction. As a result, the field gets stronger and neurons may be more likely to be activated in this area due to a higher field gradient.

Polarization (de- and/or hyperpolarization) of neural tissue may play a prominent role for both suppression of clinical symptoms, as well as induction of stimulation-induced side effects. In order to activate a neuron, the neuron has to be depolarized. Neurons may be depolarized more easily close to the cathode than by the anode (about 3-7 times more depending on type of neuron, etc.).

As illustrated, neurostimulation system 100 includes DBS probe 130 for brain applications with stimulation and/or recording electrodes 132, which may include, more than ten, more than twenty, or, for example, forty electrodes 132 provided on an outer body surface at the distal end of DBS probe 130. However, the techniques described in this disclosure are not so limited. As referred to herein, the distal end of a medical lead or probe may be the remote end of the lead with regard to the body surface area. In particular, in case of a lead for brain application, the distal end of the lead is the lower end of the lead, that is inserted deeper into the brain tissues, and which is remote to the burr-hole of the skull, through which the lead is implanted.

IPG 110 may include more than one implantable pulse generator for delivery of neurostimulation via electrodes 132, and/or one or more sensors configured to sense electrical fields within the brain of the patient, such as electrical fields representing a patient's brain activity and/or electrical fields created by delivery of DBS therapy. In examples in which IPG 110 includes both an implantable pulse generator and one or more sensors, in various examples, either the same set of electrodes or different sets of electrodes may be used for sensing as those used for delivery of DBS therapy.

In the example shown in FIG. 1, IPG 110 may be implanted within a subcutaneous pocket in the pectoral region of patient 12. IPG 110 may be surgically implanted in the chest region of a patient, such as below the clavicle or in the abdominal region of a patient. In other examples, IPG 110 may be implanted within other regions of patient 12, such as a subcutaneous pocket in the abdomen or buttocks of patient 12 or proximate to the cranium of patient 12. The neurostimulation system 100 may further include a lead extension 120 connected to IPG 110 and running subcutaneously to the skull, such as along the neck, where it terminates in a distal end that couples to a proximal end of lead 220 via one or more connectors. The proximal end of lead 220 extends from the connector to ALC 111, which in turn, couples to DBS probe 130. DBS probe 130 may be implanted in the brain tissue, for example, through a burr-hole in the skull. In some examples, ALC 111 may be located adjacent the burr-hole and external to the skull and beneath the skin. In other examples, ALC 111 may be located into a surgeon-created recess adjacent the burr-hole in the skull and/or into the burr hole itself.

Implanted lead extension 120 is coupled at one end to IPG 110 via connector block 30 (also referred to as a header), which may include, for example, electrical contacts that electrically couple to respective electrical contacts on lead extension 120. In turn, conductors of lead extension 120 are electrically coupled to the proximal end of lead 220, as set forth above. Lead 220 extends to, and comprises, the ALC 111. ALC 111 includes electronic module 500 with an active switch matrix to direct stimulation from IPG 110 to any combination of electrodes 132. Likewise, the active switch matrix electronic module 500 can direct sensing signals from any combination of electrodes 132 to IPG 110. In some examples, ALC 111 may digitize sensing signals prior to sending them to IPG 110. IPG 110 may store the sensing signals or a subset of the sensing signals, analyze the sensing signals or a subset of the sensing signals, and/or forward the sensing signals or a subset of the sensing signals to an external device via a wireless transmission.

Lead extension 120 traverses from the implant site of IPG 110, along the neck of patient 12. The distal end of lead extension 120 may connect to a proximal end of lead 220,e e.g., somewhere along the cranium of patient 12. The lead extends to ALC 111 to access brain 28. IPG 110 and ALC 111 can be constructed of biocompatible materials that resist corrosion and degradation from bodily fluids. IPG 110 may comprise a hermetic outer housing 34 to substantially enclose components, such as a processor, a therapy module, and memory. Likewise, ALC 111 may comprise a hermetic outer housing to substantially enclose electronic components.

In the example shown in FIG. 1, probe 130 is implanted within brain 28 in order to deliver electrical stimulation to one or more regions of brain 28, which may be selected based on many factors, such as the type of patient condition for which therapy system 100 is implemented to manage. Other implant sites for probe 130 and IPG 110 are contemplated. For example, IPG 110 may be implanted on or within cranium 32. As another example, probe 130 may be implanted within the same hemisphere as that shown in FIG. 1 but at multiple other target tissue sites or IPG 110 may be coupled to one or more medical leads that are implanted in one or both hemispheres of brain 28.

During implantation of probe 130 within patient 12, a clinician may attempt to position electrodes 132 of probe 130 such that electrodes 132 are able to deliver electrical stimulation to one or more target tissue sites within brain 28 to manage patient symptoms associated with a disorder of patient 12. Probe 130 may be placed at any location within brain 28 such that electrodes 132 are capable of providing electrical stimulation to target therapy delivery sites within brain 28 during treatment.

The anatomical region within patient 12 that serves as the target tissue site for stimulation delivered by system 100 may be selected based on the patient condition. Different neurological or psychiatric disorders may be associated with activity in one or more of regions of brain 28, which may differ between patients. Accordingly, the target therapy delivery site for electrical stimulation therapy delivered by probe 130 may be selected based on the patient condition. For example, a suitable target therapy delivery site within brain 28 for controlling a movement disorder of patient 12 may include one or more of the pedunculopontine nucleus (PPN), thalamus, basal ganglia structures (e.g., globus pallidus, substantia nigra or subthalamic nucleus), zona inserta, fiber tracts, lenticular fasciculus (and branches thereof), ansa lenticularis, or the Field of Forel (thalamic fasciculus). The PPN may also be referred to as the pedunculopontine tegmental nucleus.

As another example, in the case of MDD, bipolar disorder, OCD, or other anxiety disorders, probe 130 may be implanted to deliver electrical stimulation to the anterior limb of the internal capsule of brain 28, and only the ventral portion of the anterior limb of the internal capsule (also referred to as a VC/VS), the subgenual component of the cingulate cortex (which may be referred to as CG25), anterior cingulate cortex Brodmann areas 32 and 24, various parts of the prefrontal cortex, including the dorsal lateral and medial pre-frontal cortex (PFC) (e.g., Brodmann area 9), ventromedial prefrontal cortex (e.g., Brodmann area 10), the lateral and medial orbitofrontal cortex (e.g., Brodmann area 11), the medial or nucleus accumbens, thalamus, intralaminar thalamic nuclei, amygdala, hippocampus, the lateral hypothalamus, the Locus ceruleus, the dorsal raphe nucleus, ventral tegmentum, the substantia nigra, subthalamic nucleus, the inferior thalamic peduncle, the dorsal medial nucleus of the thalamus, the habenula, the bed nucleus of the stria terminalis, or any combination thereof.

As another example, in the case of a seizure disorder or Alzheimer's disease, for example, probe 130 may be implanted to deliver electrical stimulation to regions within the Circuit of Papez, such as, for example, one or more of the anterior thalamic nucleus, the internal capsule, the cingulate, the fornix, the mammillary bodies, the mammillothalamic tract (mammillothalamic fasciculus), or the hippocampus. Target therapy delivery sites not located in brain 28 of patient 12 are also contemplated.

The techniques of this disclosure may be implemented in combination with systems including smaller electrodes, such as electrodes manufactured using thin film manufacturing. Examples of such manufacturing techniques for a medical lead made from a thin film based on thin film technology are disclosed in United States Patent Application Publication No. 2011/0224765, titled, “SPIRALED WIRES IN A DEEP-BRAIN STIMULATION PROBE,” the entire contents of which are incorporated by reference herein. The thin film medical leads may be fixed on an elongated carrier to form a medical lead. These medical leads may include multiple electrode areas and may enhance the precision to address the appropriate target in the brain and relax the specification of positioning. Meanwhile, undesired side effects due to undesired stimulation of neighboring areas may be limited.

Although lead 220 and lead extension 120 are shown in FIG. 1, in other examples, probe 130 may be coupled to IPG 110 via a single lead that extends from ALC 111 to IPG 110. Moreover, although FIG. 1 illustrates system 100 as including a single probe 130 coupled to IPG 110 via lead 220, lead extension 120 and ALC 111, in some examples, system 100 may include two or more medical leads and ALCs. In some examples, each ALC may be associated with a single medical lead; in other examples, more than one medical lead may extend from an ALC. In some example, system 100 may include multiple DBS probes rather than a single DBS probe 330.

In the example shown in FIG. 1, electrodes 132 of probe 130 are shown as an array of electrodes with a complex electrode array geometry that is capable of producing shaped electrical fields. An example of a complex electrode array geometry may include an array of electrodes positioned at different axial positions along the length of a medical lead, as well as at different angular positions about the periphery, for example, circumference, of the medical lead. The complex electrode array geometry may include multiple electrodes (e.g., partial ring or segmented electrodes) around the perimeter of each medical lead 20. In other examples, the complex electrode array geometry may include electrode pads distributed axially and circumferentially about the medical lead 20. In either case, by having electrodes at different axial and angular positions, electrical stimulation may be directed in a specific direction from probe 130 to enhance therapy efficacy and reduce possible adverse side effects from stimulating a large volume of tissue. In some examples, the array of electrodes may be combined with one or more ring electrodes on probe 130.

In some examples, outer housing 34 of IPG 110 and/or the housing of ALC 111 may include one or more stimulation and/or sensing electrodes. For example, housing 34 can comprise an electrically conductive material that is exposed to tissue of patient 12 when IPG 110 is implanted in patient 12, or an electrode can be attached to housing 34.

IPG 110 and ALC 111 may deliver electrical stimulation therapy to brain 28 of patient 12 according to one or more therapy programs. A therapy program may define one or more electrical stimulation parameter values for therapy generated by a stimulation generator of IPG 110 and delivered from IPG 110 to a target therapy delivery site within patient 12 via one or more electrodes 132. The electrical stimulation parameters may define an aspect of the electrical stimulation therapy, and may include, for example, voltage or current amplitude of an electrical stimulation signal, a frequency of the electrical stimulation signal, and, in the case of electrical stimulation pulses, a pulse rate, a pulse width, a waveform shape, and other appropriate parameters such as duration or duty cycle. In addition, if different electrodes are available for delivery of stimulation, a therapy parameter of a therapy program may be further characterized by an electrode combination, which may define electrodes 132 selected for delivery of electrical stimulation and their respective polarities. In some examples, as an alternative to stimulation pulses, stimulation may be delivered using a continuous waveform and the stimulation parameters may define this waveform.

In addition to being configured to deliver therapy to manage a disorder of patient 12, therapy system 100 may be configured to sense bioelectrical brain signals of patient 12. For example, IPG 110 may include a sensing module that is configured to sense bioelectrical brain signals within one or more regions of brain 28 via a subset of electrodes 132, another set of electrodes, or both. Accordingly, in some examples, electrodes 132 may be used to deliver electrical stimulation from the therapy module to target sites within brain 28 as well as sense brain signals within brain 28. However, IPG 110 can also use a separate set of sensing electrodes to sense the bioelectrical brain signals. In some examples, the sensing module of IPG 110 may sense bioelectrical brain signals via one or more of the electrodes 132 that are also used to deliver electrical stimulation to brain 28. In other examples, one or more of electrodes 132 may be used to sense bioelectrical brain signals while one or more different electrodes of electrodes 132 may be used to deliver electrical stimulation.

Examples of bioelectrical brain signals include, but are not limited to, electrical signals generated from local field potentials (LFPs) within one or more regions of brain 28, such as, but not limited to, an electroencephalogram (EEG) signal or an electrocorticogram (ECoG) signal. In some examples, the electrical signals within brain 28 may reflect changes in electrical current produced by the sum of electrical potential differences across brain tissue.

External medical device programmer 14 is configured to wirelessly communicate with IPG 110 as needed to provide or retrieve therapy information. Programmer 14 is an external computing device that the user, for example, the clinician and/or patient 12, may use to communicate with IPG 110. For example, programmer 14 may be a clinician programmer that the clinician uses to communicate with IPG 110 and program one or more therapy programs for IPG 110. In addition, or instead, programmer 14 may be a patient programmer that allows patient 12 to select programs and/or view and modify therapy parameter values. The clinician programmer may include more programming features than the patient programmer. In other words, more complex or sensitive tasks may only be allowed by the clinician programmer to prevent an untrained patient from making undesired changes to IPG 110.

Programmer 14 may be a hand-held computing device with a display viewable by the user and an interface for providing input to programmer 14 (i.e., a user input mechanism). For example, programmer 14 may include a small display screen (e.g., a liquid crystal display (LCD) or a light emitting diode (LED) display) that presents information to the user. In addition, programmer 14 may include a touch screen display, keypad, buttons, a peripheral pointing device or another input mechanism that allows the user to navigate through the user interface of programmer 14 and provide input. If programmer 14 includes buttons and a keypad, then the buttons may be dedicated to performing a certain function, for example, a power button, the buttons and the keypad may be soft keys that change in function depending upon the section of the user interface currently viewed by the user, or any combination thereof.

In other examples, programmer 14 may be a larger workstation or a separate application within another multi-function device, rather than a dedicated computing device. For example, the multi-function device may be a notebook computer, tablet computer, workstation, cellular phone, personal digital assistant or another computing device that may run an application that enables the computing device to operate as a secure medical device programmer 14. A wireless adapter coupled to the computing device may enable secure communication between the computing device and IPG 110.

When programmer 14 is configured for use by the clinician, programmer 14 may be used to transmit programming information to IPG 110. Programming information may include, for example, hardware information, such as the type of ALC 111, the type of probe 130, the arrangement of electrodes 132 on probe 130, the position of probe 130 within brain 28, one or more therapy programs defining therapy parameter values, and any other information that may be useful for programming into IPG 110. Programmer 14 may also be capable of completing functional tests (e.g., measuring the impedance of electrodes 132 of probe 130).

With the aid of programmer 14 or another computing device, a clinician may select one or more therapy programs for therapy system 100 and, in some examples, store the therapy programs within IPG 110. Programmer 14 may assist the clinician in the creation/identification of therapy programs by providing physiologically relevant information specific to patient 12.

Programmer 14 may also be configured for use by patient 12. When configured as a patient programmer, programmer 14 may have limited functionality (compared to a clinician programmer) in order to prevent patient 12 from altering critical functions of IPG 110 or applications that may be detrimental to patient 12.

Whether programmer 14 is configured for clinician or patient use, programmer 14 is configured to communicate with IPG 110 and, optionally, another computing device, via wireless communication. Programmer 14, for example, may communicate via wireless communication with IPG 110 using radio frequency (RF) telemetry techniques known in the art. Programmer 14 may also communicate with another programmer or computing device via a wired or wireless connection using any of a variety of local wireless communication techniques, such as RF communication according to the 802.11 or BLUETOOTH® specification sets, infrared (IR) communication according to the IRDA specification set, or other standard or proprietary telemetry protocols. Programmer 14 may also communicate with other programming or computing devices via exchange of removable media, such as magnetic or optical disks, memory cards or memory sticks. Further, programmer 14 may communicate with IPG 110 and another programmer via remote telemetry techniques known in the art, communicating via a local area network (LAN), wide area network (WAN), public switched telephone network (PSTN), or cellular telephone network, for example.

Therapy system 100 may be implemented to provide chronic stimulation therapy to patient 12 over the course of several months or years. However, system 100 may also be employed on a trial basis to evaluate therapy before committing to full implantation. If implemented temporarily, some components of system 100 may not be implanted within patient 12. For example, patient 12 may be fitted with an external medical device, such as a trial stimulator, rather than IPG 110. The external medical device may be coupled to percutaneous medical leads or to implanted medical leads via a percutaneous extension. If the trial stimulator indicates DBS system 100 provides effective treatment to patient 12, the clinician may implant a chronic stimulator within patient 12 for relatively long-term treatment.

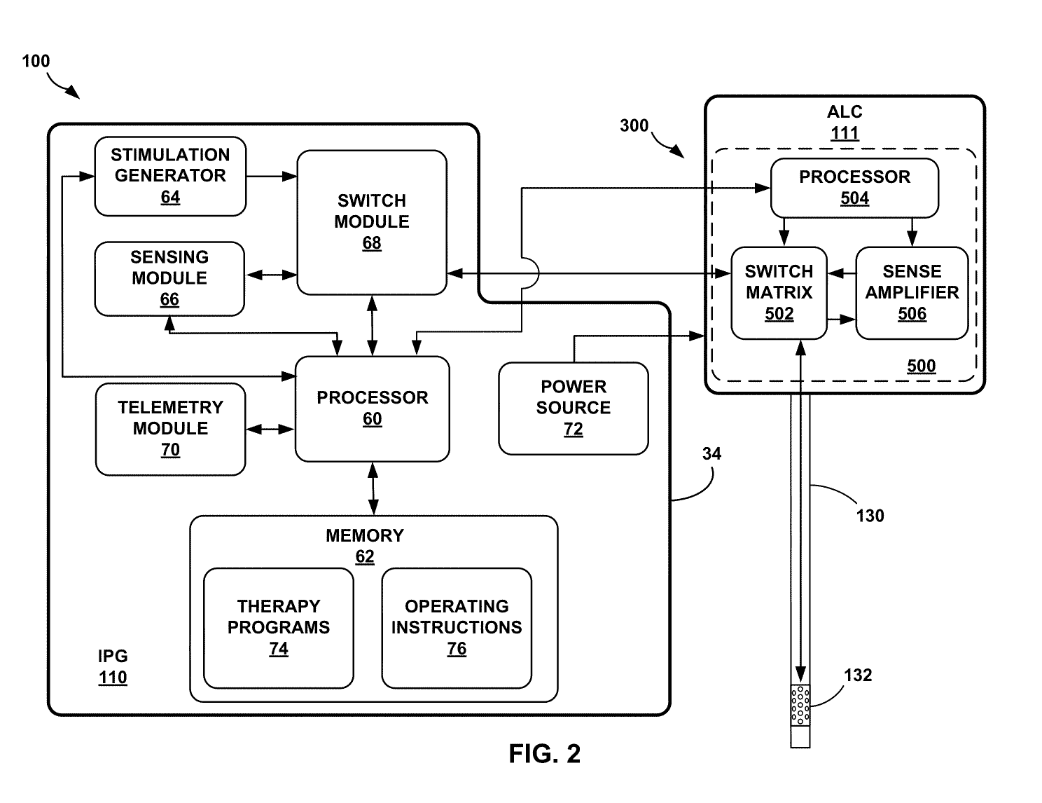

FIG. 2 is functional block diagram illustrating components of an example therapy system 100 including IMG 110 and ALC 111. In the example shown in FIG. 2, IPG 110 includes processor 60, memory 62, stimulation generator 64, sensing module 66, switch module 68, telemetry module 70, and power source 72. Memory 62, as well as other memories described herein, may include any volatile or non-volatile media, such as a random access memory (RAM), read only memory (ROM), non-volatile RAM (NVRAM), electrically erasable programmable ROM (EEPROM), flash memory, and the like. Memory 62 may store computer-readable instructions that, when executed by processor 60, cause IPG 110 to perform various functions described herein.

In the example shown in FIG. 2, memory 62 stores therapy programs 74 and operating instructions 76, for example, in separate memories within memory 62 or separate areas within memory 62. Each stored therapy program 74 defines a particular program of therapy in terms of respective values for electrical stimulation parameters, such as an electrode combination, current or voltage amplitude, and, if stimulation generator 64 generates and delivers stimulation pulses, the therapy programs may define values for a pulse width, and pulse rate of a stimulation signal. The stimulation signals delivered by IPG 110 may be of any form, such as stimulation pulses, continuous-wave signals (e.g., sine waves), or the like. Operating instructions 76 guide general operation of IPG 110 under control of processor 60, and may include instructions for monitoring brain signals within one or more brain regions via electrodes 132 and delivering electrical stimulation therapy to patient 12.

Stimulation generator 64, under the control of processor 60, generates stimulation signals for delivery to patient 12 via selected combinations of electrodes 132. In some examples, stimulation generator 64 generates and delivers stimulation signals to one or more target regions of brain 28 (FIG. 1), via a select combination of electrodes 132, based on one or more stored therapy programs 74. Processor 60 selects the combination of electrodes 132 with control signals to processor 504 of ALC 111. In turn, processor 504 of ALC 111 selectively activates active switch matrix 504 to direct the stimulation signals received from stimulation generator 64 to the selected electrodes 132. The stimulation parameter values and target tissue sites within brain 28 for stimulation signals or other types of therapy may depend on the patient condition for which therapy system 100 is implemented to manage.

The processors described in this disclosure, including processor 60 and processor 504, may include one or more digital signal processors (DSPs), general-purpose microprocessors, application specific integrated circuits (ASICs), field programmable logic arrays (FPGAs), or other equivalent integrated or discrete logic circuitry, or combinations thereof. The functions attributed to processors described herein may be provided by a hardware device and embodied as software, firmware, hardware, or any combination thereof. Processor 60 is configured to control stimulation generator 64 according to therapy programs 74 stored by memory 62 to apply particular stimulation parameter values specified by one or more therapy programs.

Processor 60 may control switch module 68 to select stimulation generator 64 or sensing module 66. In turn, processor 60 directs processor 504 of electronic module 500 to apply the stimulation signals generated by stimulation generator 64 to selected combinations of electrodes 132, or to sense signals from selected combinations of electrodes 132 via sense amplifier 506 of electronic module 500. In particular, active switch matrix 502 of electronic module 500 may couple stimulation signals to selected conducting tracks within probe 130, which, in turn, deliver the stimulation signals to selected electrodes 132. Hence, although there may be many, for example, 40, electrodes, active switch matrix 502 may select a subset of one, two or more electrodes for delivery of stimulation pulses. Active switch matrix 502 may be a switch array, an array of one or more transistors such as Field-Effect Transistors (FETs) switch matrix, multiplexer, and/or demultiplexer, or any other type of switching module configured to selectively couple stimulation energy to selected electrodes 132 and to selectively sense bioelectrical brain signals with selected electrodes 132. Hence, stimulation generator 64 is coupled to electrodes 132 via switch module 68, conductors between IPG 110 and ALC 111, active switch matrix 502, and conducting tracks within probe 130. Additionally, the logic path between stimulation generator and electrodes 132 may include one or more discrete components such as capacitors, resistors, logic gates, transistors, and the like. Thus, it will be understood that when reference is made to coupling of stimulation generator 64 or other components of IPG 110 to electrodes 132, this refers to the enabling of a logic path between the logic components so that signals may be transferred there between, and is not intended to necessarily require a direct electrical coupling of the components.

In some examples, however, IPG 110 does not include switch module 68 and all switching functions may be performed by active switch matrix 502. For example, IPG 110 may include multiple sources of stimulation energy (e.g., current sources). Stimulation generator 64 may be a single channel or multi-channel stimulation generator. In particular, stimulation generator 64 may be capable of delivering a single stimulation pulse, multiple stimulation pulses or continuous signal at a given time via a single electrode combination or multiple stimulation pulses at a given time via multiple electrode combinations. In some examples, however, stimulation generator 64 and active switch matrix 502 may be configured to deliver multiple channels on a time-interleaved basis. For example, active switch matrix 502 may serve to time divide the output of stimulation generator 64 across different electrode combinations at different times to deliver multiple programs or channels of stimulation energy to patient 12.

In addition to, or instead of stimulation generator 64 of IPG, a stimulation generator may reside within ALC (not shown) and may generate the stimulation pulses that are routed to electrodes 132 via active switch matrix 502. In such cases, the stimulation generator within the ALC may receive power from power source 72 and may receive control signals from stimulation generator 64 or other logic of IPG 110. The stimulation generator in ALC may be provided in addition to, or instead of, stimulation generator 64 of IPG 110. Thus, electronics for driving probe 130 and electrodes 132 of lead may reside in IPG 110, ALC 111, or some combination thereof. As is the case with any stimulation generator 64 of IPG, any stimulation of ALC may be a single channel or multi-channel stimulation generator as set forth above.

Sensing module 66, under the control of processor 60, is configured to sense bioelectrical brain signals of patient 12 via active switch matrix 502, sense amplifier 506, and a selected subset of electrodes 132 or with one or more electrodes 132 and at least a portion of a conductive outer housing 34 of IPG 110, at least a portion of a conductive outer housing of ALC 111, an electrode on outer housing 34 of IPG 110, an electrode on an outer housing of ALC 111, or another reference. Processors 60 and 504 may control switch module 68 and active switch matrix 502 to electrically connect sensing module 66 to selected electrodes 132 via active switch matrix 502 and sense amplifier 506 of ALC 111. In this way, sensing module 66 may selectively sense bioelectrical brain signals with different combinations of electrodes 132.

Telemetry module 70 is configured to support wireless communication between IPG 110 and an external programmer 14 or another computing device under the control of processor 60. Processor 60 of IPG 110 may receive, as updates to programs, values for various stimulation parameters from programmer 14 via telemetry module 70. The updates to the therapy programs may be stored within therapy programs 74 portion of memory 62. Telemetry module 70 in IPG 110, as well as telemetry modules in other devices and systems described herein, such as programmer 14, may accomplish communication by RF communication techniques. In addition, telemetry module 70 may communicate with external medical device programmer 14 via proximal inductive interaction of IPG 110 with programmer 14. Accordingly, telemetry module 70 may send and receive information to/from external programmer 14 on a continuous basis, at periodic intervals, or upon request from IPG 110 or programmer 14.

Power source 72 delivers operating power to various components of IPG 110. Power source 72 may include a small rechargeable or non-rechargeable battery and a power generation circuit to produce the operating power. Recharging may be accomplished through proximal inductive interaction between an external charger and an inductive charging coil within IPG 110. In some examples, power requirements may be small enough to allow IPG 110 to utilize patient motion and implement a kinetic energy-scavenging device to trickle charge a rechargeable battery. In other examples, traditional batteries may be used for a limited period of time.

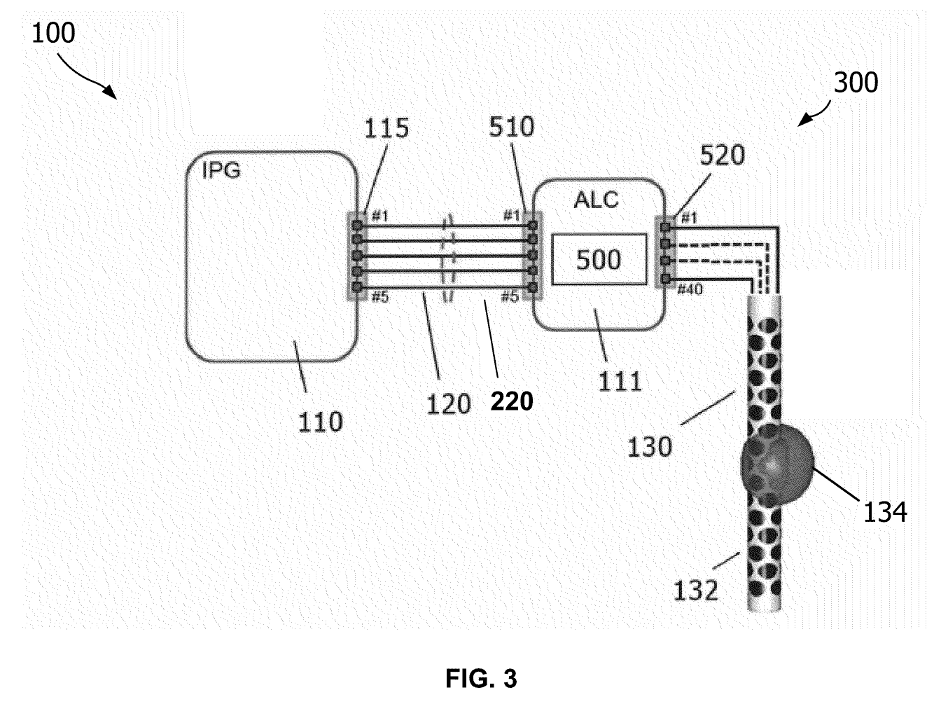

FIG. 3 is a functional block diagram illustrating electrical connections between IPG 110, ALC 111 and DBS probe 130 within neurostimulation system 100. As illustrated in FIG. 3, IPG 110 connects lead extension 120 via connectors 115. Connectors at the distal end of lead extension 120 may couple to connectors at the proximal end of lead 220. In one example, lead 220 comprises ALC 111 and DBS probe 130. More specifically, a proximal end of lead 220 may extend to ALC 111 which, in turn, may connect to DBS probe 130. DBS probe 130 may comprise a separate conductor for each of electrodes 132 which are routed via connector 520. ALC 111 includes electronic module 500 with an active switch matrix 502 (FIG. 2) to direct stimulation signals from IPG 110 to any combination of electrodes 132 and/or direct sensed signals from electrodes 132 to IPG 110.

In the illustration of FIG. 3, example stimulation/sensing zone 134 is depicted. Stimulation/sensing zone 134 utilizes a subset of electrodes 132 for stimulation or sensing. Active switch matrix 502 of electronic module 500 may be used to select any combination of electrodes for stimulation and sensing functionality. In some examples, active switch matrix 502 of electronic module 500 within ALC 111 can connect any number of the available electrodes to any number of one or more stimulation signals or ground, such that stimulation is not limited to being provided across pairs of two of electrodes 132. In this manner, other stimulation and sensing zones may be activated using different subsets of electrodes and/or by using field steering techniques such as varying the resistance of paths in multi-electrode combinations, anodal shielding and other field steering techniques. As set forth above, the stimulation signals may be generated by voltage-controlled or current-controlled logic such a stimulation generator 64 that resides within IPG 110, within ALC, or a combination thereof.

In the example configuration of FIG. 3, lead extension 120/lead 220 and connectors 115, 510 provide five conductive paths between IPG 110 and ALC 111. IPG 110 has a N-pin connector 115 (e.g., N=5) which is connected via the lead extension 120 and lead 220 with the 5-pin connector 510 (or N-pin connector 510) of ALC 111. In the example of IPG 110 and ALC 111, the five conductors between IPG 110 and ALC 111 may include a power conductor, a ground conductor, a communication conductor, a conductor for a first pulse generator within IPG 110, and a conductor for a second pulse generator within IPG 110. The control line may provide instructions from IPG 110 for directing stimulation pulses to selected electrodes via active switch matrix 502 of electronic module 500 or providing sensing connectivity between electrodes and IPG 110 via active switch matrix 502 of electronic module 500. In some examples, the power conductor may serve a dual purpose of providing clock or timing information between IPG 110 and ALC 111. For example, the voltage over the power conductor may be sent as a square wave or other periodic signal. In some examples, the timing information provided by the power conductor may be used to coordinate sensing and stimulation functions as isolating sensing circuitry, such as sense amplifier 506 (FIG. 2) in ALC 111, from the stimulation generators may be required to protect the sensing circuitry from the stimulation pulse. Any number of conductors may be provided in the alternative, with the conductors serving similar or different functions to those set forth above.

As described above, ALC 111 includes a N-pin connector 510, which is configured to be coupled to respective conductive paths of proximal end of lead 220. ALC may also include a M-pin connector 520 (e.g., M=40) for DBS probe 130, e.g., for electrically coupling respective electrodes 132 to electronic module 500. As shown in FIG. 3, in some examples, N is greater than M. It is mechanically possible to design these two feedthrough connectors 510, 520 with a high pin density to reduce the area of ALC 111 significantly. However, this area advantage may only materialize if the electrical components of ALC 111 are shrunk in similar proportions as the feedthrough connectors 510, 520. Moreover, a very thin ALC 111, most desirable to reduce its impact on skin erosion, may need a high pin density, but also a reduction in the height of both connectors 510, 520 and interior electrical components. Thus, both the electronics volume and area of ALC 111 are miniaturized to realize a small ALC 111. Note that techniques to shrink ALC 111 can also be applied to the IPG 110, or any other implant module, for example, to trade for an increase in battery life and/or increased functionality. In some examples, M=N or M is greater than N.

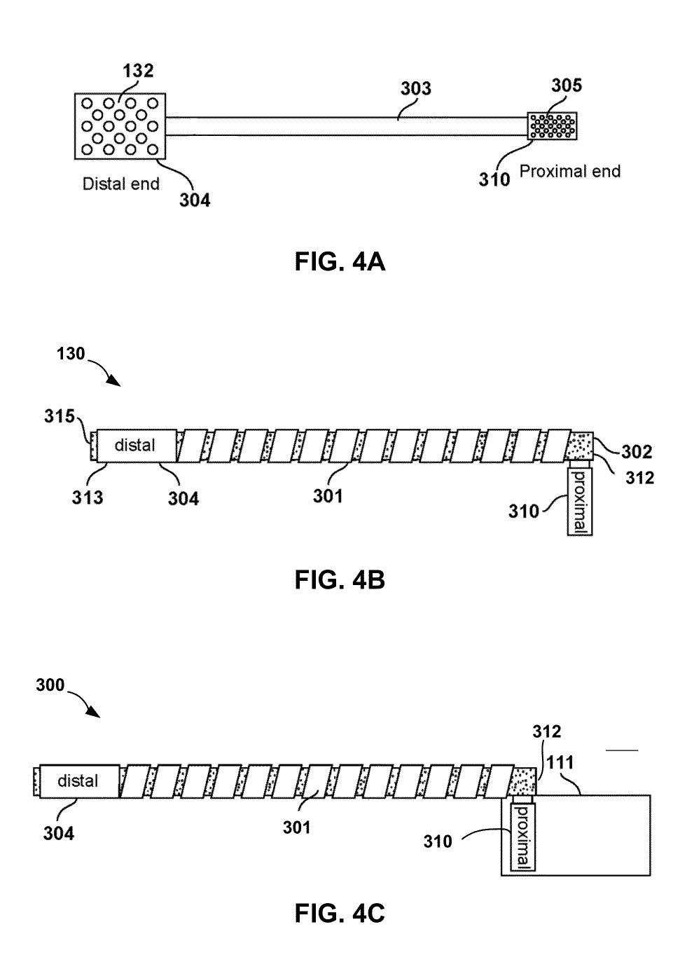

FIGS. 4A-4C illustrate examples of medical leads for stimulation and/or sensing. FIG. 4C further illustrates a typical architecture for an assembly including DBS probe 130 and ALC 111. ALC 111 includes an active switch matrix and electronics to address electrodes 132 on distal end 304 of thin film 301. Electrodes 132 are arranged at distal end 312 and next to distal tip 315 of DBS probe 130, as illustrated in FIGS. 4A and 4B.

DBS probe 130 comprises an elongated carrier 302 for thin film 301, where elongated carrier 302 provides the mechanical configuration of DBS probe 130 and thin film 301. Elongated carrier 302 may be a flexible carrier, such as a flexible tubing. In some examples, elongated carrier 302 may be formed from a silicon tubing.

Elongated carrier 302 may have any suitable configuration. In some examples, elongated carrier 302 may be an elongated member having a circular cross-section, although other cross-sections are contemplated, such as, e.g., square or hexagonal. Elongated carrier 302 may be a solid member or have a hollow core. In some examples, it is preferred that elongated carrier 302 be relatively stiff during implantation but able to flex or bend to some degree after implantation. The hollow core may allow for the insertion of a stiffening member such as a stylet into the hollow core, e.g., during implantation of lead 300. Elongated carrier 302 may be configured to not substantially shrink, stretch, or compress during and/or after implantation.

In some examples, elongated carrier 302 should be flexible and have a good rotational torque transfer, e.g., in instances of permanent (chronic) implant of lead 300. Some acute applications may have a different set of preferences. For instance, in acute implantation, no burr-hole devise may be used and flexibility and limited compressibility are of less concern.

Elongated carrier 302 may be formed of any suitable material including silicone, titanium, and/or polyether ether ketone (PEEK) based materials. For the mechanical requirements as mentioned above, other polymers can be more useful e.g. bionate. In addition, metal tubes (e.g., laser machined to bendable chains) may be used. In acute applications, a solid metal may be used for elongated carrier 302. In acute application, there may not be a need for elongated carrier 302 to be hollow or flexible. In chronic applications, elongated carrier 302 is implanted with a stiffener inside. After implantation, the stiffener may be removed.

Distal portion of lead 300 may have a diameter between about 0.5 millimeters (mm) and about 3 mm diameter, e.g., about 1.3 mm. The diameter of lead 300 may be defined by the diameter of carrier core 302 in combination with the thickness of thin film 301 and any coating applied over carrier core 302 and/or thin film 301. The proximal portion of lead 300 (the portion adjacent to ALC 111) may have a diameter between about 0.5 mm and about 4mm diameter. The length of lead 300 may be about 10 centimeters (cm) to about 20 cm, e.g., about 15 cm, and may vary based on the particular application, e.g., acute versus chronic implantation. Other dimensions than those examples described herein are contemplated.

Thin film 301 may include at least one electrically conductive layer, such as one made of a biocompatible material. Thin film 301 may be formed by a thin film product having a distal end 304, a cable 303 with conducting tracks and a proximal end 310, as illustrated in FIG. 4A.

Thin film structures may provide an advantage that small structures may be built of with this technology. A thin film is a layer or multilayer structure of material ranging from fractions of a nanometer (monolayer) to several micrometers in thickness. Electronic semiconductor devices and optical coatings may be the main applications benefiting from thin-film construction. Thin film technology and thin film manufacturing processes may allow the manufacturing of leads for medical purposes such as neurostimulation leads like, for example, DBS leads with diameters of less than 2 mm, for example 0.75 mm to 1.50 mm and a plurality of electrodes, such as 40 electrodes, although any number of electrodes may be used, including more than 40 electrodes. In addition, thin film technology allows for various configurations of high density electrode arrangements, including, for example, a series of small ring electrodes or an arrangement of electrodes with more complex geometries. During stimulation or sensing, different combinations of electrodes may be used to precisely direct the stimulation or sensing within a patient.

As illustrated in FIG. 4B, thin film 301 is attached to elongated carrier 302 and further processed to constitute DBS probe 130. For example, thin film 301 may be wrapped around elongated carrier 302 in a helical fashion and all or a portion of thin film 301 may be attached to elongated carrier 302, for example, by gluing or otherwise adhering thin film 301 to elongated carrier 302 via an adhesive. Additionally or alternatively, a thin coating may be formed over thin film 301 after being wrapped around elongated carrier 302 to secure thin film 301 to elongated carrier 302.

As illustrated in FIG. 4C, proximal end 310 of thin film 301 arranged at proximal end 312 of DBS probe 130 is electrically connected to ALC 111. ALC 111 comprises the active switch matrix 502 of the DBS steering electronics. Distal end 304 comprises electrodes 132 for the brain stimulation. Proximal end 310 comprises interconnect contacts 305 for each individual conducting track in cable 303. Cable portion 303 comprises conducting tracks (not shown) defined by thin film 301 to connect each of distal electrodes 132 to a designated proximal contact 305. For example, an individual electrode 132 at the distal end 304 of thin film 301 may be electrically coupled to an individual interconnect contact 305 located at the proximal end 310 of thin film via an individual conductive track extending between the electrode 132 and contact 305. Each of the individual tracks of thin film 301 may be electrically isolated from each other. Electrodes 132, which may include a relatively large number of electrodes provide an array of electrodes on the distal end of probe 130. The array of electrodes provides fine adjustment capabilities for sensing and stimulation with lead 300.

In other examples, a DBS lead may include, for example, four 1.5 millimeters wide cylindrical electrodes, at the distal end spaced by between about 0.5 millimeters and 1.5 millimeters. In this example, a diameter of the medical lead may be about 1.27 millimeters and the metal used for the electrodes and the interconnect wires may be an alloy of platinum and iridium. The coiled interconnect wires may be insulated individually by fluoropolymer coating and protected in an 80 micron urethane tubing. With such an electrode design, the current distribution may emanate uniformly around the circumference of the electrode, which medical leads to stimulation of all areas surrounding the electrode.

As compared to probe 130, such a design may limit fine spatial control over stimulation field distributions. The lack of fine spatial control over field distributions implies that stimulation easily spreads into adjacent structures inducing adverse side effects in about thirty percent of the patients. To overcome this problem, medical leads with high-density electrode arrangements, such as those examples illustrated herein, facilitate electrical field position adjustments in smaller increments, hence providing the ability to steer the stimulation field to the appropriate target.

The clinical benefit of DBS may be largely dependent on the spatial distribution of the stimulation field in relation to brain anatomy. To improve efficacy and efficiency of DBS while avoiding unwanted side effects, precise control over the stimulation field is important. Electrodes 132 of probe 130, with high-density electrode arrangements, provide much greater adjustability and precision than a medical lead with cylindrical electrodes.

Thin film structures used to form the leads with high-density electrode arrangements may be relatively fragile, and the handling of the leads may be difficult. Also, the connection of the thin film with the electronics of the overall system is important, as this connection should be mechanically strong and electrically reliable. Due to the mechanical properties of the thin film, this connection forms an ambitious challenge. It is therefore an object of the present disclosure to provide a medical lead system and a method of manufacturing a medical lead system, especially in that the fixation of thin film 301, e.g., to ALC 11, can be made mechanically strong and electrically reliable.

Connecting thin film 301 at proximal end 310 of probe 130 to ALC 111 requires forming electrical connections between proximal contacts 305 corresponding to one or more conductive tracks defined by thin film 301 and electrical connector 520 of ALC 111 as well as providing a durable mechanical connection between probe 130 and ALC 111 to facilitate a reliable electrical and mechanical connection. In some examples, probe 130 may be electrically and mechanically connected to ALC 111 by way of a landing block that may be used to make a reliable connection. The “landing block,” a thin metal frame, provides both a mechanical fixation of the flexible tube as well as an electrical connection to ALC 111.

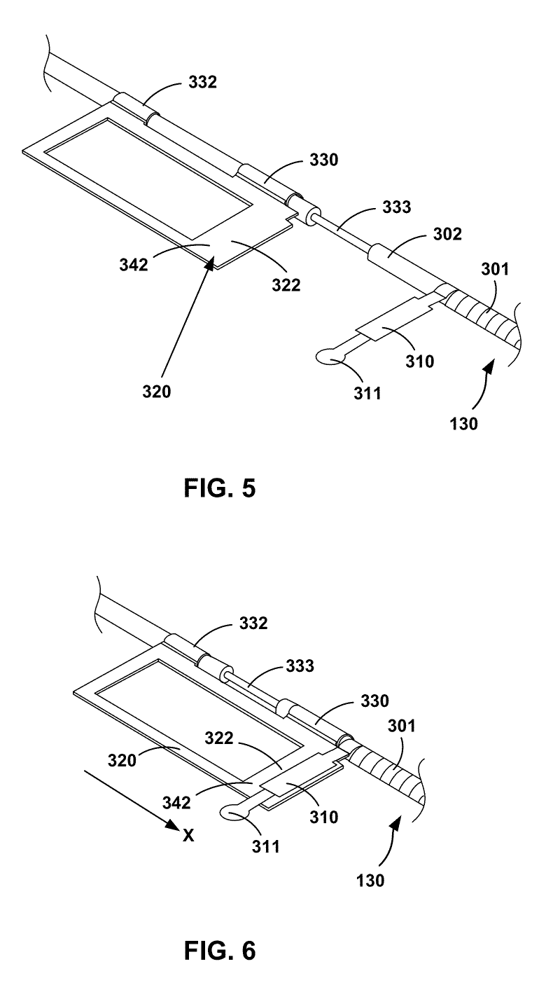

FIGS. 5-11 illustrate a proximal portion of medical lead 300 during manufacturing of medical lead 300, including probe 130 and ALC 111 of medical lead 300. More specifically, FIGS. 5-11 illustrate forming the connection between proximal end 310 of thin film 301 of probe 130 and ALC 111 of medical lead 300. Medical lead 300 may be suitable for use in DBS system 100 as generally described above with respect to FIGS. 1-3.

As shown in FIG. 5, elongated carrier 302 is provided as a part of a probe 130 and thin film 301 is helically wound around elongated carrier 302 to form probe 130. Proximal end 310 of thin film 301 is not wound around elongated carrier 302 but projects away in the region of the proximal end of elongated carrier 302 in a substantially tangential direction relative to probe 130. Proximal end 310 of thin film 301 is configured to be supported by fixation zone 322 of frame element 320. The substantially tangential arrangement of fixation zone 322 relative to the direction of probe 130 may advantageous, among others, since thin film 301, which is wound around elongated carrier 302, may provide fixation zone 322 with a smooth bend such that the curvature of thin film 301 should not be abruptly altered.

Proximal end 310 of thin film 301 is mechanically connected to ALC 111 by way of frame element 320. Frame element 320 provides fixation zone 322 for proximal end 310 of thin film 301. Proximal end 310 carries interconnect contacts 305 (not shown in FIGS. 5 and 6) of the thin film 301. Frame element 320 comprises plate portion 342 and fixation zone 322 is on a surface of plate portion 342. Proximal end 310 of thin film 301 may be attached at fixation zone 322 and stabilized by plate portion 342. For example, plate portion 342 may be a flat or planar plate portion. A planar fixation zone 322 facilitates a stable and reliable fixation of thin film 301 and support of proximal end 310 of thin film 301. The configuration of frame element 320 allows the overall structure of medical lead 300 to have acceptably small dimensions to facilitate implantation into a mammalian body, like the skull of a patient to be treated with DBS.

The fixation between fixation zone 322 and proximal end 310 of thin film 301 is established, for example, by gluing using an adhesive or other suitable options. By this, the fixation of a thin film 301 can be made mechanically strong and electrically reliable as the fixation is done within a zone and thus over a certain area and not by, for example, multiple dot-like connections. Removable tab 311 may be used to provide tension on thin film 301 during the winding of the thin film 301 around the carrier 302 and/or during the fixation of proximal end 310 on fixation zone 322 of frame element 320. Following the fixation of proximal end 310 on fixation zone 322 of frame element 320, removable tab 311 may be removed. Frame element 320 may be formed from a metal plate and may have and suitable dimensions, e.g., a thickness between 0.1 mm to 1 mm. This aspect allows an improved forming of the frame element and a good stability and support for thin film 301.

Frame element 320 has two mounting portions 330, 332, wherein first mounting portion 330 is mounted to elongated carrier 302 and second mounting portion 332 is also mounted to elongated carrier 302, after frame element 320 is partially slid along the axis X (labelled in FIG. 6) of elongated carrier 302 from the configuration shown in FIG. 5. In the example as shown in FIGS. 5-11, mounting portions 330, 332 of frame element 320 are mounting tube portions, which extend entirely around elongated carrier 302. Alternatively, mounting portions 330, 332 may be mounting tube segment portions, which partially extend around elongated carrier 302.

In addition, post 333 may add stability to elongated carrier 302 adjacent mounting portions 330, 332 and proximal end 310 of thin film 301. For example, elongated carrier 302 may be a flexible tube, such as a silicon tube. Post 333 may run within a hollow center of carrier 302 to stiffen a portion of elongated carrier 302 and facilitate securing frame element 320 to elongated carrier 302 by clamping mounting portions 330, 332 on the proximal end of elongated carrier 302, thereby pinching the flexible tubing between post 333 and mounting portions 330, 332. In addition, post 333 may extend past the proximal end 310 of thin film 301 to provide dimensional stability to elongated carrier 302 adjacent frame element 320 and proximal end 310 of thin film 301. This may protect thin film 301 from bending adjacent to adjacent frame element 320

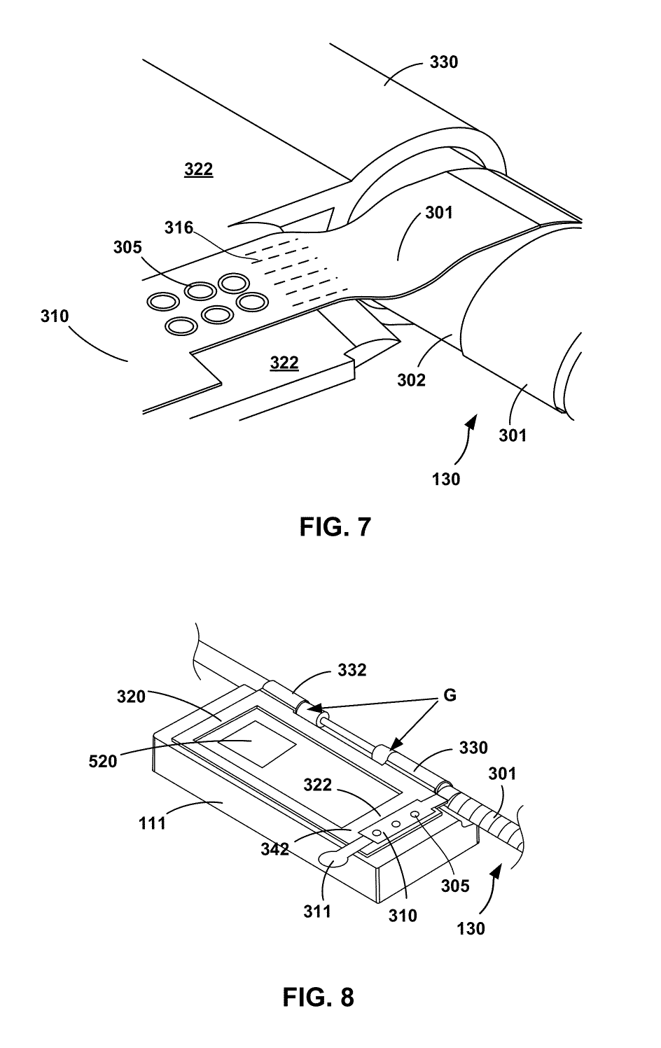

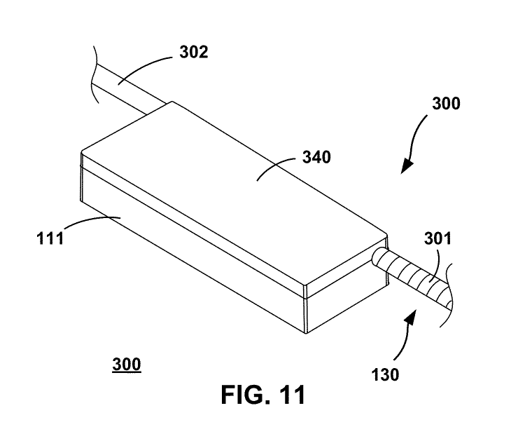

FIG. 7 illustrates a close-up of the connection between proximal end 310 of thin film 301 and fixation zone 322 of frame element 320. Also illustrated in FIG. 7 are contact pads 305, which, in addition to be electrically coupled to individual electrodes 132 via conductive tracks 316 of thin film 301, may provide mechanically support to thin film 301 by being compressed with the application of lid 340 to ALC 111 (FIG. 11). By way of mounting portions 330, 332, a reliable and stable mounting of frame element 320 to elongated carrier 302 may be provided. Thus, a stable and reliable connection of elongated carrier 302, frame element 320 and thin film 301 is provided. As indicated in FIG. 8, the fixation between mounting portions 330, 332 and elongated carrier 302 can be established by suitable attachment means and is here exemplarily established by gluing with adhesive G. Further options may additionally or alternatively include form-fit options, welding, overmolding, fixation pins, etc.

Mounting portions 330, 332 may include at least one mounting tube portion or at least one mounting tube segment portion, which is at least partially extends around elongated carrier 302. Mounting portions 330, 332 may be formed from a plate portion of frame element 301. In other examples, mounting portions 330, 332 may be replaced with a plurality of mounting fingers, which are at least partially extended around the flexible tube. The mounting fingers may allow a lightweight and stable connection. Also, in case that the mounting finger shall be fixated to the underlying flexible tube by glue, such a connection may be provided with a consistent and homogenous glue portion.

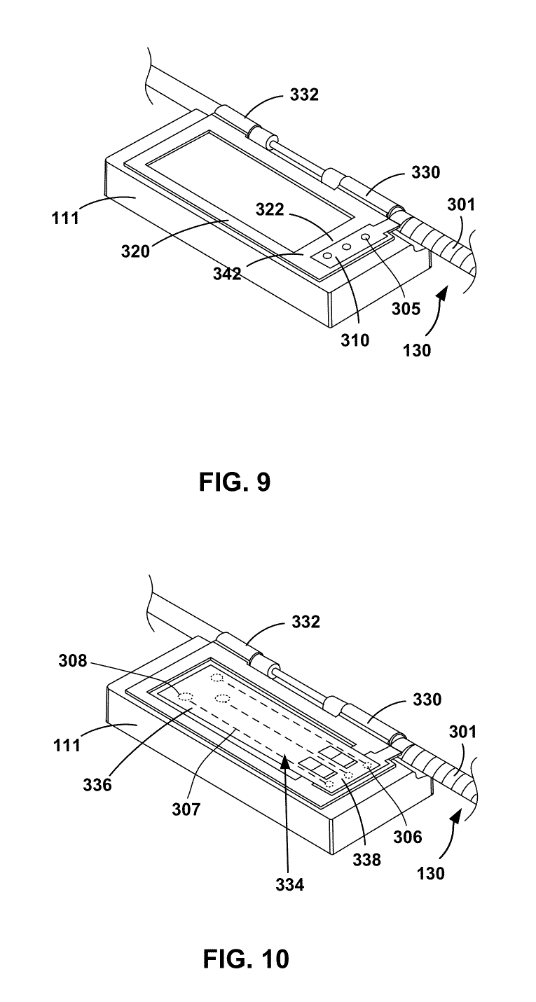

As best seen in FIG. 9, after fixation of thin film 301 to frame element 320 and the fixation of frame element 320 to elongated carrier 302, an ALC 111 is provided and ALC 111 is mounted to frame element 320, for example, by welding. ALC 111 may contain at least a part of electronics of medical probe 130, such as a switch matrix. In some cases, ALC 111 may include one or more stimulation generators as discussed above. As shown in FIG. 9, proximal end 310 of thin film 301 includes contacts 305 (only three are shown in FIG. 9 for ease of illustration), where each contact may correspond to a conductive track (e.g., tracks 316 partially shown in FIG. 7) and electrode of array 132. Example contacts 305 are also illustrated in FIG. 7 along with the partial representation of tracks 316. Although not shown, each individual track 316 of thin film 301 may extend from an individual contact 305 at proximal end 310 to an individual electrode 132 located distal to proximal end 310 of thin film, e.g., at distal end 304.

As shown in FIG. 10, interposer connector 334 has a first connection portion 336 for the connection of the relatively large connectors 520 (FIG. 3) of ALC 111 and a second connection portion 338 for the connection to the contacts 305 (e.g., being smaller than connectors 520 of ALC 111) and conductive tracks (e.g., tracks 316 conceptually shown in FIG. 7) of thin film 301. Portion 338 of interposer 334 includes electrical contacts 306 that electrically couple to respective ones of the proximal contacts 305 (FIG. 4A/FIG. 9) of thin film 301. For example, the arrangement of electrical contacts 306 of interposer may be substantially the same as the arrangement of contacts 305 at distal end of thin film 301. When properly aligned the second connection portion 338 may be placed over the distal end 310 to electrically couple the contacts 306 of interposer 334 with the contacts 305 of thin film 301.

Each electrical contact 306 of portion 338 of interposer 334 is electrically coupled through a respective conductive trace 307 (or path) to a respective individual electrical contact 308 of portion 336. The electrical contacts 308 of portion 336 may couple to an individual contact of connector 520 of ALC 111. For example, the arrangement of electrical contacts 308 of interposer may be substantially the same as the arrangement of contacts (not shown) of connector 520 of ALC. When properly aligned the first connection portion 336 may be placed over electrical connector 520 to electrically couple the contacts 308 of interposer 334 with the contacts of connector 520. The electrical contacts of portion 336 may have a different pitch, size and/or other configuration in one embodiment than those of electrical contacts of portion 338. Each electrical contact may be attached another contact using any suitable technique, such as, e.g., a conductive polymer adhesive.

In this manner, interposer 334 may electrically couple the individual contacts 305 at the distal end 310 of thin film 301 to individual contacts of connector 520 of ALC 11. In such a configuration, the individual electrodes 132 at the distal end 304 of thin film 301 may be electrically coupled to ALC 111. When electrically coupled in such manner, electrical signals may be conducted from ALC 11 to electrodes 132, e.g., to delivery electrical stimulation and/or sense electrical signals.

Interposer connector 334 may improve the stability and reliability of the connection between connectors 520 of ALC 111 and contacts 305 of thin film 301. For example, connectors 520 of ALC 111, such as, e.g., in the form of feedthroughs, may be larger than contacts 305 of thin film 301. With interposer connector 334, no large area is required to realize the interconnection between interconnect contacts 305 of thin film 301 and the relatively large connectors 520 of ALC 111.

Although not shown, in an alternative to the example shown in FIG. 10, rather than interposer connector being a separate member, interposer connector 334 can be also formed by a prolongation of the thin film and can have a first connection portion 336 for the connection of the relatively large connectors 520 of ALC 111 and a second connection portion 338 for the connection to the connectors (being smaller than connectors 520 of ALC 111) and the tracks of the thin film 301.

As shown in FIG. 11, after the assembly of interposer connector 334, lid 340 may be attached, which covers interposer connector 334, frame element 320 and proximal end 310 of thin film 301 as fixed on fixation zone 322.

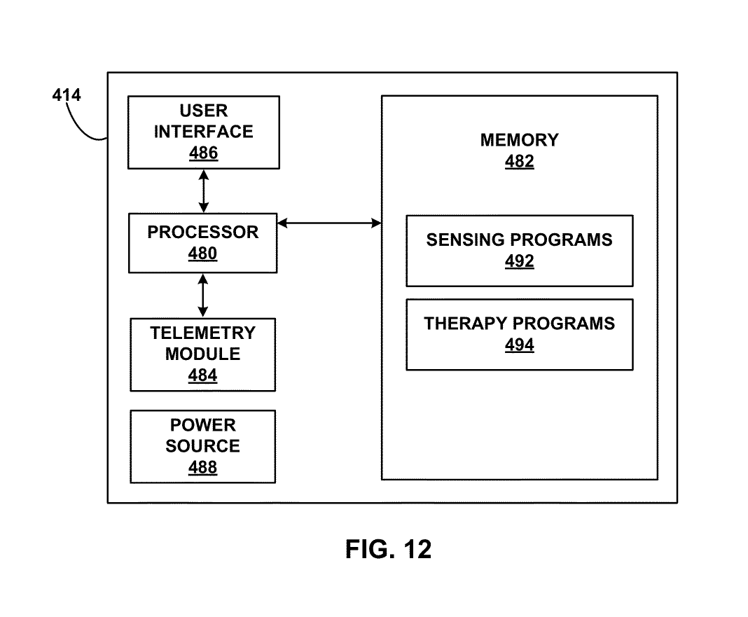

FIG. 12 is a functional block diagram illustrating components of an example medical device programmer 414. Programmer 414 includes processor 480, memory 482, telemetry module 484, user interface 486, and power source 488. Processor 480 controls user interface 486 and telemetry module 484, and stores and retrieves information and instructions to and from memory 482. Programmer 414 may be configured for use as a clinician programmer or a patient programmer. Processor 480 may comprise any combination of one or more processors including one or more microprocessors, DSPs, ASICs, FPGAs, or other equivalent integrated or discrete logic circuitry. Accordingly, processor 480 may include any suitable structure, whether in hardware, software, firmware, or any combination thereof, to perform the functions ascribed herein to processor 480 and programmer 414.

A user, such as a clinician or patient 12, may interact with programmer 414 through user interface 486. User interface 486 includes a display (not shown), such as a LCD or LED display or other type of screen, with which processor 480 may present information related to the therapy (e.g., therapy programs). In addition, user interface 486 may include an input mechanism to receive input from the user. The input mechanisms may include, for example, any one or more of buttons, a keypad (e.g., an alphanumeric keypad), a peripheral pointing device, a touch screen, or another input mechanism that allows the user to navigate through user interfaces presented by processor 480 of programmer 414 and provide input. In other examples, user interface 486 also includes audio circuitry for providing audible notifications, instructions or other sounds to patient 12, receiving voice commands from patient 12, or both.

Memory 482 may include instructions for operating user interface 486 and telemetry module 484, and for managing power source 488. Processor 480 may store the therapy programs and in memory 482 as stored therapy programs 494 and store the sensing parameters and the recorded results of the sensing as stored sensing programs 492. A clinician may review the stored therapy programs 494 and stored sensing programs 492 (e.g., during programming of IPG 110) to select one or more therapy programs with which IPG 110 may deliver efficacious electrical stimulation to patient 12. For example, the clinician may interact with user interface 486 to retrieve the stored therapy programs 494 and stored sensing programs 492.

In some examples, processor 480 is configured to generate and present, via a display of user interface 486, a graphical user interface (GUI) that presents a list of therapy programs. A user (e.g., a clinician) may interact with the GUI to manipulate the list of therapy programs. In some examples, a user may also interact with the graphical user interface to select a particular therapy program, and, in response to receiving the user input, programmer 414 may provide additional details about the therapy program. For example, the additional details presented by programmer 414 may include details about the individual parameter settings of the therapy program, such as the electrical stimulation parameter values, electrode combination, or both.

In some examples, patient 12, a clinician or another user may interact with user interface 486 of programmer 414 in other ways to manually select programs from the stored therapy programs 494 and stored sensing programs 492 for programming IPG 110, generate new therapy and sensing programs, modify stored therapy programs 494 and stored sensing programs 492, transmit the selected, modified, or new programs to IPG 110, or any combination thereof.

Memory 482 may include any volatile or nonvolatile memory, such as RAM, ROM, EEPROM or flash memory. Memory 482 may also include a removable memory portion that may be used to provide memory updates or increases in memory capacities. A removable memory may also allow sensitive patient data to be removed before programmer 414 is used by a different patient.

Wireless telemetry in programmer 414 may be accomplished by RF communication or proximal inductive interaction of external programmer 414 with IPG 110. This wireless communication is possible through the use of telemetry module 484. Accordingly, telemetry module 484 may be similar to the telemetry module contained within IPG 110. In other examples, programmer 414 may be capable of infrared communication or direct communication through a wired connection. In this manner, other external devices may be capable of communicating with programmer 414 without needing to establish a secure wireless connection.

Power source 488 is configured to deliver operating power to the components of programmer 414. Power source 488 may include a battery and a power generation circuit to produce the operating power. In some examples, the battery may be rechargeable to allow extended operation. In other examples, traditional batteries (e.g., nickel cadmium or lithium ion batteries) may be used. In addition, programmer 414 may be directly coupled to an alternating current outlet to operate.

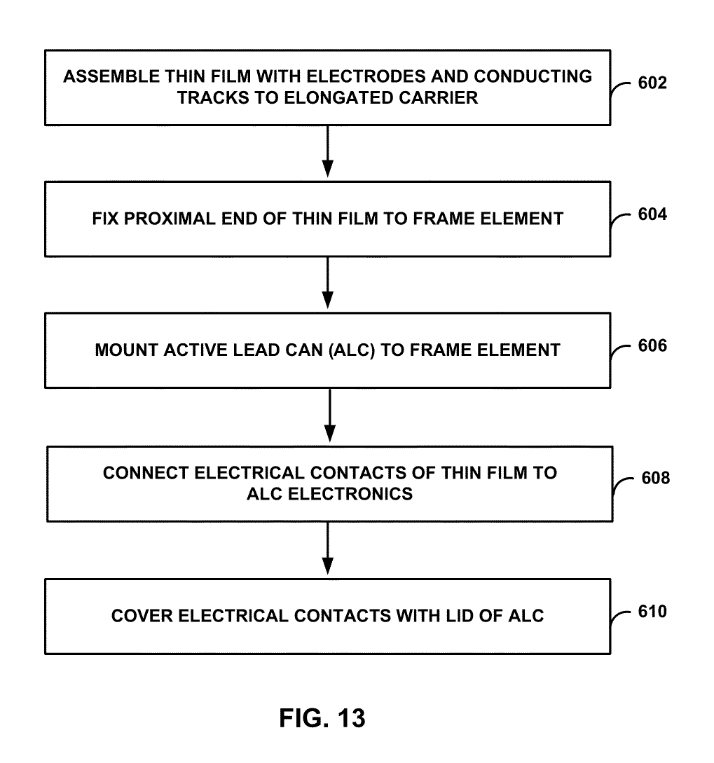

FIG. 13 is a flowchart illustrating example techniques for manufacturing a medical lead. For clarity, the techniques of FIG. 13 are described with respect to medical lead 300, as illustrated in FIGS. 3-11.

Thin film 301 is attached to elongated carrier 302 (602) to form probe 130 (FIG. 5). In some examples, assembly of thin film 301 on elongated carrier 302 includes winding thin film 301 on elongated carrier 302 in a helical fashion.

As shown in FIG. 8, proximal end 310 of thin film 301, which includes proximal contacts 305, is fixed to fixation zone 322 of frame element 320 (604). Removable tab 311 may be used to provide tension on thin film 301 during the winding and during the fixation of proximal end 310 on fixation zone 322 of frame element 320. Following the fixation of proximal end 310 on fixation zone 322 of frame element 320, removable tab 311 may be removed.

ALC 111 is mounted to frame element 320 (606). For example, ALC 111 may be welded to frame element 320. Then, an electrical connection is formed between the electronics module 500 of ALC 111 and proximal contacts 305 of thin film 301 (608). For example, contacts 306 and 308 of interposer connector 334 may provide for a connection between proximal contacts 305 of thin film 301 and connectors 520 (FIG. 3) of ALC 111 to provide electrical connection paths 307 between the proximal contacts 305 of thin film 301 and connectors 520. In one embodiment, an additional set of feedthroughs of ALC 111 may be integrally-coupled to conductors carried by a proximal end of lead 220. The connector at the proximal end of lead 220 couples these conductors via respective conductors of lead extension 130 to one or more IPGs 110 (FIG.3). In an alternative embodiment, ALC 111 may have a connector that can be removably connected directly to lead extension 120, which in turn may electrically connect ALC 111 to one or more remotely located pulse generators, such as those of IPG 110. In this alternative embodiment, proximal end of lead 220 is eliminated, and the lead includes only probe 130 and ALC.

As shown in FIG. 11, proximal contacts 305 of thin film 301 may be covered with lid 340 of ALC 111 (610). Lid 340 may be secured by welding or by other suitable techniques.

While the techniques described herein are suitable for systems and methods involving DBS therapies, and may be used treat such disorders as Parkinson's disease, Alzheimer's disease, tremor, dystonia, depression, epilepsy, OCD, and other disorders, the techniques are not so limited. One or more such techniques and systems may be applied to treat disorders such as chronic pain disorders, urinary or fecal incontinence, sexual dysfunction, obesity, mood disorders, gastroparesis or diabetes, and may involve other types of stimulation such as spinal cord stimulation, cardiac stimulation, pelvic floor stimulation, sacral nerve stimulation, peripheral nerve stimulation, peripheral nerve field stimulation, gastric stimulation, or any other electrical stimulation therapy. In some cases, the electrical stimulation may be used for muscle stimulation.

In addition, it should be noted that examples of the systems and techniques described herein may not be limited to treatment or monitoring of a human patient. In alternative examples, example systems and techniques may be implemented in non-human patients, e.g., primates, canines, equines, pigs, and felines. These other animals may undergo clinical or research therapies that my benefit from the subject matter of this disclosure.

The techniques of this disclosure may be implemented in a wide variety of computing devices, medical devices, or any combination thereof. Any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

The disclosure contemplates computer-readable storage media comprising instructions to cause a processor to perform any of the functions and techniques described herein. The computer-readable storage media may take the example form of any volatile, non-volatile, magnetic, optical, or electrical media, such as a RAM, ROM, NVRAM, EEPROM, or flash memory that is tangible. The computer-readable storage media may be referred to as non-transitory. A server, client computing device, or any other computing device may also contain a more portable removable memory type to enable easy data transfer or offline data analysis. The techniques described in this disclosure, including those attributed to various modules and various constituent components, may be implemented, at least in part, in hardware, software, firmware or any combination thereof. For example, various aspects of the techniques may be implemented within one or more processors, including one or more microprocessors, DSPs, ASICs, FPGAs, or any other equivalent integrated or discrete logic circuitry, as well as any combinations of such components, remote servers, remote client devices, or other devices. The term “processor” or “processing circuitry” may generally refer to any of the foregoing logic circuitry, alone or in combination with other logic circuitry, or any other equivalent circuitry.

Such hardware, software, firmware may be implemented within the same device or within separate devices to support the various operations and functions described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

The techniques described in this disclosure may also be embodied or encoded in an article of manufacture including a computer-readable storage medium encoded with instructions. Instructions embedded or encoded in an article of manufacture including a computer-readable storage medium , may cause one or more programmable processors, or other processors, to implement one or more of the techniques described herein, such as when instructions included or encoded in the computer-readable storage medium are executed by the one or more processors. Example computer-readable storage media may include random access memory (RAM), read only memory (ROM), programmable read only memory (PROM), erasable programmable read only memory (EPROM), electronically erasable programmable read only memory (EEPROM), flash memory, a hard disk, a compact disc ROM (CD-ROM), a floppy disk, a cassette, magnetic media, optical media, or any other computer readable storage devices or tangible computer readable media. The computer-readable storage medium may also be referred to as storage devices.

In some examples, a computer-readable storage medium comprises non-transitory medium. The term “non-transitory” may indicate that the storage medium is not embodied in a carrier wave or a propagated signal. In certain examples, a non-transitory storage medium may store data that can, over time, change (e.g., in RAM or cache).

Various examples have been described herein. Any combination of the described operations or functions is contemplated. These and other examples are within the scope of the following claims.

What is claimed is:

1. A medical device system for at least one of delivery of electrical stimulation pulses or sensing of physiological signals, the system comprising:
an elongated carrier;
a thin film attached to the elongated carrier, the thin film including a plurality of electrodes, a plurality of electrical contacts, and a plurality of conducting tracks, each of the plurality of conducting tracks providing an electrical connection between at least one of the plurality of electrodes and one of the plurality of electrical contacts; and
a frame element including a fixation zone for the plurality of electrical contacts of the thin film.

2. The system of claim 1, wherein the thin film is wound on the elongated carrier in a helical fashion.

3. The system of claim 1, wherein the fixation zone is substantially tangential to an outer cylindrical surface of the elongated carrier.

4. The system of claim 1, wherein the frame element includes a plate portion, wherein the fixation zone is on a surface of the plate portion.

5. The system of claim 1, wherein the frame element includes a mounting portion with which the frame element is mounted on the elongated carrier.

6. The system of claim 5, wherein the mounting portion includes at least one mounting tube portion or at least one mounting tube segment portion, which is at least partially wound around the elongated carrier.

7. The system of claim 1, further comprising an active lead can including a switch matrix and a substantially sealed housing containing the switch matrix, wherein the frame element is mounted to the active lead can.

8. The system of claim 7, wherein the active lead can further includes feedthroughs extending through the substantially sealed housing, the system further comprising an interposer that provides electrical connection paths between the plurality of electrical contacts of the thin film and the feedthroughs.

9. The system of claim 7, further comprising an implantable medical device coupled to the active lead can via a lead.

10. The system of claim 9, wherein N electrical connection tracks are defined between the implantable medical device and the active lead can, and wherein N is less than a total number of individual electrodes of the plurality of electrodes.

11. The system of claim 10, wherein the active lead can includes a pulse generator.

12. The system of claim 10, wherein the implantable medical device includes a pulse generator.

13. The system of claim 1, wherein a proximal end of the thin film is electrically coupled to an interposer and affixed to the fixation zone of the frame element.

14. The system of claim 1, wherein a proximal end of the thin film is not wrapped around the elongated carrier.

15. A method of manufacturing a medical lead, the method comprising:
assembling a thin film to an elongated carrier, the thin film including a plurality of electrodes, a plurality of electrical contacts, and a plurality of conducting tracks, each of the plurality of conducting tracks providing an electrical connection between at least one of the plurality of electrodes and one of the plurality of electrical contacts; and
fixing the plurality of electrical contacts of the thin film on a fixation zone of a frame element.

16. The method of claim 15, wherein assembling the thin film to the elongated carrier comprises winding the thin film on the elongated carrier in a helical fashion.

17. The method of claim 15, further comprising mounting an active lead can to the frame element, the active lead can including a switch matrix and a substantially sealed housing containing the switch matrix.

18. The method of claim 17, wherein the active lead can further includes feedthroughs extending through the substantially sealed housing, the method further comprising connecting an interposer between the plurality of electrical contacts of the thin film and the feedthroughs to provide electrical connection paths between the plurality of electrical contacts of the thin film and the feedthroughs.

19. The method of claim 17, further comprising connecting a cable to a connector of the active lead can to electrically connect the medical lead to one or more remotely located stimulation pulse generators, wherein the switch matrix is configured to selectively couple each of the electrodes to the stimulation pulse generators.

20. A medical device system comprising:
an implantable medical device including a first substantially sealed housing;
an active lead can including a second substantially sealed housing operatively coupled to the implantable medical device; and
a medical lead extending from the active lead can and operatively coupled to the active lead can, wherein the medical lead includes:
an elongated carrier;
a thin film attached to the elongated carrier, the thin film including a plurality of electrodes, a plurality of electrical contacts, and a plurality of conducting tracks, each of the plurality of conducting tracks providing an electrical connection between at least one of the plurality of electrodes and one of the plurality of electrical contacts; and
a frame element including a fixation zone for the plurality of electrical contacts of the thin film.
